(19) United States
(12) Patent Application Publication
(10) Pub. No.: US 2021/0030350 A1
(43) Pub. Date: Feb. 4, 2021

(54) METHODS OF DETECTING NEURODEGENERATIVE DISEASE

(71) Applicant: Washington University, St. Louis, MO (US)

(72) Inventors: Rajendra Apte, St. Louis, MO (US); Gregory Van Stavern, St. Louis, MO (US)

(73) Assignee: Washington University, St. Louis, MO (US)

(21) Appl. No.: 16/971,747

(22) PCT Filed: Feb. 21, 2019
(86) PCT No.: PCT/US19/18926

§ 371 Date: Aug. 21, 2020

Related U.S. Application Data
(60) Provisional application No. 62/634,002, filed on Feb. 22, 2018.

(51) Int. Cl. A61B 5/00; A61B 3/10; A61K 51/04; G01N 33/68

(52) U.S. Cl. CPC A61B 5/4088; A61B 3/102; A61K 51/0455; G01N 2800/2814; G01N 33/6896; G01N 2333/4709; G01N 2800/2821; A61K 51/0453

(57) ABSTRACT

Among the various aspects of the present disclosure is the provision of a method for detecting neurodegenerative diseases, disorders, or conditions. Briefly, the present disclosure is directed to a non-invasive method for measuring foveal area and thickness, which has been shown to correlate with the detection of biomarkers used in the detection of neurodegenerative diseases such as Alzheimer's disease and clinical dementia.

### CROSS-REFERENCE TO RELATED APPLICATIONS

This application claims priority from U.S. Provisional Application Ser. No. 62/634,002 filed on 22 Feb. 2018, which is incorporated herein by reference in its entirety.

### STATEMENT REGARDING FEDERALLY SPONSORED RESEARCH OR DEVELOPMENT

Not applicable.

### FIELD OF THE INVENTION

The present disclosure generally relates to methods of noninvasive imaging for detection of disease.

### BACKGROUND OF THE INVENTION

Alzheimer's disease (AD) is marked by slowly progressive memory loss, behavioral changes, and deterioration of executive function. Symptoms of AD only become apparent after irreversible neuron loss has already occurred. Biomarkers for AD have been identified but are invasive and expensive. Optical coherence tomography (OCT) and OCT angiography (OCTA) are noninvasive imaging techniques allowing for analysis of retinal and microvascular anatomy. Here, OCT and OCTA technology are used to compare retinal architecture and vascularization between cognitively normal individuals with pre-clinical, biomarker positive AD and biomarker negative age-matched controls.

### SUMMARY OF THE INVENTION

Among the various aspects of the present disclosure is the provision of a method for detecting neurodegenerative disease.

One aspect of the present disclosure provides for a method of identifying a subject at risk for developing or at risk for having a neurodegenerative disease.

In some embodiments, the method comprises measuring a foveal avascular zone (FAZ) area or an inner foveal thickness, an outer foveal thickness, or a total foveal thickness.

In some embodiments, the neurodegenerative disease causes vascular or retinal abnormalities in an eye of the subject.

In some embodiments, the neurodegenerative disease is an amyloid-β-associated neurodegenerative disease.

In some embodiments, the subject does not exhibit cognitive dysfunction.

In some embodiments, if FAZ area is increased compared to a control or standard or the inner foveal thickness, the outer foveal thickness, or the total foveal thickness is decreased compared to a control or standard, the subject is identified as being at risk for developing or having a neurodegenerative disease, wherein the control or standard is obtained from a subject not having a preclinical neurodegenerative disease.

In some embodiments, if the measured FAZ area is greater than about 0.3 mm², the subject is identified as being at risk for developing or at risk for having a neurodegenerative disease.

In some embodiments, if the inner foveal thickness is less than about 73 μm, the subject is identified as being at risk for developing or at risk for having a neurodegenerative disease.

In some embodiments, if the outer foveal thickness is less than 190 μm, the subject is identified as being at risk for developing or at risk for having a neurodegenerative disease.

In some embodiments, if the total foveal thickness is less than about 260 μm, the subject is identified as being at risk for developing or at risk for having a neurodegenerative disease.

In some embodiments, the measuring of the foveal avascular zone (FAZ) area or inner, outer, or total foveal thickness is performed using optical coherence tomography (OCT) or optical coherence tomography angiography (OCTA).

In some embodiments, the neurodegenerative disease is an amyloid-β-associated neurodegenerative disease, preclinical Alzheimer's disease, or dementia.

In some embodiments, the neurodegenerative disease is preclinical Alzheimer's disease.

In some embodiments, the subject is administered early therapeutic intervention to treat or prevent neuronal loss or brain atrophy.

In some embodiments, the method comprises obtaining a CSF sample from the subject, wherein the CSF sample comprises increased levels of Aβ-42 and tau protein compared to a control or a standard, wherein the control or standard is obtained from a subject not having a predinical neurodegenerative disease.

In some embodiments, the method comprises administering a PET imaging agent to a subject selected from Pittsburgh compound and Florbetapir ¹⁸F-AV-45 compound and detecting the PET imaging agent using PET.

Another aspect of the present disclosure provides for a method of detecting a preclinical neurodegenerative disease in a subject comprising measuring a foveal avascular zone (FAZ) area or measuring an inner foveal thickness, an outer foveal thickness, or a total foveal thickness.

In some embodiments, the preclinical neurodegenerative disease causes vascular or retinal abnormalities in an eye of the subject.

In some embodiments, the predinical neurodegenerative disease is an amyloid-β-associated neurodegenerative disease.

In some embodiments, the subject does not exhibit cognitive dysfunction.

In some embodiments, an increased FAZ area compared to a control or standard or a decrease of the inner foveal thickness, the outer foveal thickness, or the total foveal thickness, compared to a control or standard, indicates detection of a preclinical neurodegenerative disease, wherein the control or standard is obtained from a subject not having a preclinical neurodegenerative disease.

In some embodiments, an FAZ area greater than about 0.3 mm² indicates detection of a predinical neurodegenerative disease.

In some embodiments, an inner foveal thickness less than about 73 μm indicates detection of a preclinical neurodegenerative disease.

In some embodiments, an outer foveal thickness less than 190 μm indicates detection of a preclinical neurodegenerative disease.

In some embodiments, a total foveal thickness less than about 260 μm indicates detection of a predinical neurodegenerative disease.

In some embodiments, the measuring of the foveal avascular zone (FAZ) area or inner, outer, or total foveal thickness is performed using optical coherence tomography (OCT) or optical coherence tomography angiography (OCTA).

In some embodiments, the preclinical neurodegenerative disease is a predinical amyloid-β-associated neurodegenerative disease, preclinical Alzheimer's disease, or preclinical dementia.

In some embodiments, the preclinical neurodegenerative disease is preclinical AD.

In some embodiments, the subject is administered early therapeutic intervention to treat or prevent neuronal loss or brain atrophy.

In some embodiments, the method comprises obtaining a CSF sample from the subject, wherein the CSF sample comprises increased levels of Aβ-42 and tau protein compared to a control or standard, wherein the control or standard is obtained from a subject not having a preclinical neurodegenerative disease.

In some embodiments, the method comprises administering a PET imaging agent to a subject selected from Pittsburgh compound and Florbetapir ¹⁸F-AV-45 compound and detecting the PET imaging agent using PET.

In some embodiments, the control or standard is selected from measurements from (i) a subject having been administered a PET imaging agent selected from Pittsburgh compound and Florbetapir ¹⁸F-AV-45 compound and detecting the PET imaging agent using PET or (ii) a subject having CSF analysis of Aβ42 protein level and the subject was PET-negative or Aβ42-negative

Briefly, therefore, the present disclosure is directed to a non-invasive imaging method for the detection of neurodegenerative diseases such as Alzheimer's disease.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a series of images showing the Foveal Avascular Zone (FAZ) Measurements. Measurements were obtained using optical coherence tomography (OCT) angiography (Avanti OptoVue; OptoVue). Top images depict the angiogram with nonflow areas of 0.212 mm² (A) and 0.311 mm² (B); bottom images, OCT scans.

FIG. 2 is a series of box and whisker plots showing Foveal Thickness and Foveal Avascular Zone (FAZ) Measurements. Data are shown as box and whisker plots, where whiskers represent 1.5 times the interquartile range. A, Positron emission tomography (PET) imaging results are shown for fluorine 18-labeled florbetapir compound testing. Open circles indicate outliers. B, Cerebrospinal fluid (CSF) analysis results are shown for β-amyloid 42 and τ protein biomarkers. C and D, Participants with negative findings for all biomarkers (PET and/or CSF) were compared with those with positive findings for at least 1 test.

FIG. 3 is a Receiver Operating Characteristics Curve for Foveal Avascular Zone (FAZ). The receiver operating characteristics curve shows sensitivities (true-positive rate) and specificities (false-positive rate) of the FAZ comparison between all participants with biomarker-positive and biomarker-negative findings. Area under the curve is 0.8007 (95% CI, 0.6647-0.9367). Lower CI limits were also calculated for the data point closest to the nondiscriminatory (diagonal) line, assuming a normal distribution and a binomial distribution of the data.

### DETAILED DESCRIPTION OF THE INVENTION

The present disclosure is based, at least in part, on the discovery that the foveal avascular zone (FAZ) area was significantly increased in Alzheimer's disease-biomarker-positive patients and inner foveal thickness was decreased in biomarker positive patients. Described herein is a noninvasive detection of changes in retinal vasculature and thickness in cognitively normal, Alzheimer's disease-biomarker-positive patients (without any dementia) using optical coherence tomography (OCT) and optical coherence tomography angiography (OCTA).

Conventional knowledge has previously thought that neuronal changes precede vascular changes in AD, where the presently disclosed findings are counterintuitive in demonstrating significant changes in the retinal vasculature prior to onset of dementia in biomarker positive AD. Furthermore, it has never been shown before the presently disclosed study that, in cognitively normal AD patients that do not have dementia, there are any changes in the retinal vasculature or retinal neurons. All studies in neurodegenerative diseases have previously focused on patients with dementia or mild to moderate cognitive impairment.

As shown herein, patients with biomarker positive, pre-clinical (asymptomatic) Alzheimer's disease (AD) have retinal microvascular abnormalities in addition to foveal thinning. Furthermore, patients with pre-clinical AD can be identifiable by OCTA characteristics prior to the onset of cognitive dysfunction, which would allow for early therapeutic intervention to prevent further neuronal loss.

Optical coherence tomography (OCT) and optical coherence tomography angiography (OCTA) can offer a noninvasive, cost-efficient and rapid means to screen individuals for pre-clinical Alzheimer's disease, and may better identify individuals for whom more expensive and invasive biomarker testing is justified.

Neurodegenerative Disease

The compositions and methods as described herein can be used to detect, diagnose, or treat a neurodegenerative disease, disorder, or condition (including preclinical neurodegenerative diseases, disorders, or conditions). For example, the present disclosure provides for the detection, diagnosis, or treatment of neurodegenerative diseases that present in the eye. As another example, the present disclosure provides for the detection, diagnosis, or treatment of neurodegenerative diseases that are amyloid-β-associated neurodegenerative diseases. As another example, the present disclosure provides for the detection of any vascular or neuronal abnormalities in neurodegenerative diseases (e.g., Alzheimer's disease) that are manifested in the eye.

As described herein, the studies used the presence of amyloid-β biomarkers as a biomarker for preclinical Alzheimer disease.

The disclosed method can be useful in detecting amyloid-β-associated diseases, disorders, or conditions. Amyloid-β has been implicated in many neurodegenerative diseases, disorders, and conditions (Maltsev et al., Ageing Res Rev. 2011 September; 10(4):440-52). Amyloid-β-associated diseases, disorders, and conditions can include amyloid diseases, Parkinson's disease, polyglutamine diseases such as Huntington's Disease (HD), Alzheimer's disease, prion diseases, Down syndrome, vascular dementia, multiple system atrophy, amyotrophic lateral sclerosis, or epilepsy.

Amyloid-β-associated diseases, disorders, and conditions can include diseases caused by abnormal protein aggregation. Abnormal protein aggregation characterizes many, if not all, neurodegenerative disorders, not just AD and Parkinson's disease, but also Creutzfeldt-Jakob disease, motor neuron diseases, the large group of polyglutamine disorders, including Huntington's disease, as well as diseases of peripheral tissue like familial amyloid polyneuropathy (FAP).

Tests for biomarker status, as used herein, have especially high negative predictive value in assessing the risk of developing clinically detectable AD.^{10,12,13} In addition, both tests have been validated in long-term longitudinal studies to estimate onset of clinical dementia,¹⁴ such that positive findings for either test is considered diagnostic of preclinical AD.¹¹ Although these methods are useful in assessing individuals at risk for AD, they are expensive, time-consuming, invasive, and difficult to implement in routine clinical screening and care.

For example, a neurodegenerative disease, disorder, or condition can be Alzheimer's disease (AD). AD is the most common cause of dementia characterized by progressive memory loss, behavioral changes, and dysfunction of speech, language, and perception. It is currently believed that amyloid Beta (Aβ) plaques and neurofibrillary tangles (NFTs) lead to neuronal loss and brain atrophy. There is also evidence of vascular dysfunction and chronic hypoperfusion in AD patients.

As another example, a neurodegenerative disease can be amyotrophic lateral sclerosis (ALS), Alexander disease, Alpers' disease, Alpers-Huttenlocher syndrome, alpha-methylacyl-CoA racemase deficiency, Andermann syndrome, Arts syndrome, ataxia neuropathy spectrum, ataxia (e.g., with oculomotor apraxia, autosomal dominant cerebellar ataxia, deafness, and narcolepsy), autosomal recessive spastic ataxia of Charlevoix-Saguenay, Batten disease, beta-propeller protein-associated neurodegeneration, Cerebro-Oculo-Facio-Skeletal Syndrome (COFS), Corticobasal Degeneration, CLN1 disease, CLN10 disease, CLN2 disease, CLN3 disease, CLN4 disease, CLN6 disease, CLN7 disease, CLN8 disease, cognitive dysfunction, congenital insensitivity to pain with anhidrosis, dementia, familial encephalopathy with neuroserpin inclusion bodies, familial British dementia, familial Danish dementia, fatty acid hydroxylase-associated neurodegeneration, Gerstmann-Straussler-Scheinker Disease, GM2-gangliosidosis (e.g., AB variant), HMSN type 7 (e.g., with retinitis pigmentosa), Huntington's disease, infantile neuroaxonal dystrophy, infantile-onset ascending hereditary spastic paralysis, Huntington's disease (HD), infantile-onset spinocerebellar ataxia, juvenile primary lateral sclerosis, Kennedy's disease, Kuru, Leigh's Disease, Marinesco-Sjdgren syndrome, Mild Cognitive Impairment (MCI), mitochondrial membrane protein-associated neurodegeneration, Motor neuron disease, Monomelic Amyotrophy, Motor neuron diseases (MND), Multiple System Atrophy, Multiple System Atrophy with Orthostatic Hypotension (Shy-Drager Syndrome), multiple sclerosis, multiple system atrophy, neurodegeneration in Down's syndrome (NDS), neurodegeneration of aging, Neurodegeneration with brain iron accumulation, neuromyelitis optica, pantothenate kinase-associated neurodegeneration, Opsoclonus Myoclonus, prion disease, Progressive Multifocal Leukoencephalopathy, Parkinson's disease (PD), PD-related disorders, polycystic lipomembranous osteodysplasia with sclerosing leukoencephalopathy, prion disease, progressive external ophthalmoplegia, riboflavin transporter deficiency neuronopathy, Sandhoff disease, Spinal muscular atrophy (SMA), Spinocerebellar ataxia (SCA), Striatonigral degeneration, Transmissible Spongiform Encephalopathies (Prion Diseases), or Wallerian-like degeneration.

Neurodegenerative Disease Biomarkers

As described herein, optical coherence tomography (OCT) and optical coherence tomography angiography (OCTA) can offer a noninvasive, cost-efficient and rapid means to screen individuals for pre-clinical neurodegenerative disease (NDD) (e.g., Alzheimer's disease), and can better identify individuals for whom more expensive and invasive biomarker testing is justified. As such, a subject with significantly increased foveal avascular zone (FAZ) area or decreased inner foveal thickness (e.g., compared to a control or a standard) can receive further testing or early interventional therapy. As such, conventional diagnostics or detection of biomarkers can be used after the disclosed OCT and OCTA tests. Such conventional neurodegenerative disease treatment and diagnoses processes are known in the art (see e.g., F1000Research 2018, 7(F1000 Faculty Rev):1161). Except as otherwise noted herein, therefore, the process of further testing and therapy can be carried out in accordance with such processes. For example, Positron Emission Tomography (PET) imaging using Pittsburgh compound or ¹⁸F-AV-45 compound can be used. Furthermore, CSF sample testing for increased Aβ-42 and Tau protein can be used as a biomarker for NDDs, such as Aβ-associated NDDs. However, both modalities are time consuming, expensive, and invasive.

In some embodiments, a foveal avascular zone (FAZ) area can be measured. For example, the foveal avascular zone (FAZ) area can be greater than about 0.1 mm², greater than about 0.15 mm², greater than about 0.2 mm², greater than about 0.25 mm², greater than about 0.3 mm², greater than about 0.35 mm², greater than about 0.4 mm², greater than about 0.45 mm², greater than about 0.5 mm², greater than about 0.55 mm², greater than about 0.6 mm², or greater than about 0.65 mm². As another example, the foveal avascular zone (FAZ) area can be greater than about 0.3 mm². As another example, the FAZ can be greater than any value between about 0.1 mm² and about 0.6 mm². Recitation of each of these discrete values is understood to include ranges between each value. Recitation of each range is understood to include discrete values within the range.

In some embodiments, an inner foveal thickness can be measured. For example, the inner foveal thickness can be less than about 1 μm; less than about 2 μm; less than about 3 μm; less than about 4 μm; less than about 5 μm; less than about 6 μm; less than about 7 μm; less than about 8 μm; less than about 9 μm; less than about 10 μm; less than about 11 μm; less than about 12 μm; less than about 13 μm; less than about 14 μm; less than about 15 μm; less than about 16 μm; less than about 17 μm; less than about 18 μm; less than about 19 μm; less than about 20 μm; less than about 21 μm; less than about 22 μm; less than about 23 μm; less than about 24 μm; less than about 25 μm; less than about 26 μm; less than about 27 μm; less than about 28 μm; less than about 29 μm; less than about 30 μm; less than about 31 μm; less than about 32 μm; less than about 33 μm; less than about 34 μm; less than about 35 μm; less than about 36 μm; less than about 37 μm; less than about 38 μm; less than about 39 μm; less than about 40 μm; less than about 41 μm; less than about 42 μm; less than about 43 μm; less than about 44 μm; less than about 45 μm; less than about 46 μm; less than about 47 μm; less than about 48 μm; less than about 49 μm; less than about 50 μm; less than about 51 μm; less than about 52 μm; less than about 53 μm; less than about 54 μm; less than about 55 μm; less than about 56 μm; less than about 57 μm; less than about 58 μm; less than about 59 μm; less than about 60 μm; less than about 61 μm; less than about 62 μm; less than about 63 μm; less than about 64 μm; less than about 65 μm; less than about 66 μm; less than about 67 μm; less than about 68 μm; less than about 69 μm; less than about 70 μm; less than about 71 μm; less than about 72 μm; less than about 73 μm; less than about 74 μm; less than about 75 μm; less than about 76 μm; less than about 77 μm; less than about 78 μm; less than about 79 μm; less than about 80 μm; less than about 81 μm; less than about 82 μm; less than about 83 μm; less than about 84 μm; less than about 85 μm; less than about 86 μm; less than about 87 μm; less than about 88 μm; less than about 89 μm; less than about 90 μm; less than about 91 μm; less than about 92 μm; less than about 93 μm; less than about 94 μm; less than about 95 μm; less than about 96 μm; less than about 97 μm; less than about 98 μm; less than about 99 μm; less than about 100 μm; less than about 101 μm; less than about 102 μm; less than about 103 μm; less than about 104 μm; less than about 105 μm; less than about 106 μm; less than about 107 μm; less than about 108 μm; less than about 109 μm; less than about 110 μm; less than about 111 μm; less than about 112 μm; less than about 113 μm; less than about 114 μm; less than about 115 μm; less than about 116 μm; less than about 117 μm; less than about 118 μm; less than about 119 μm; less than about 120 μm; less than about 121 μm; less than about 122 μm; less than about 123 μm; less than about 124 μm; less than about 125 μm; less than about 126 μm; less than about 127 μm; less than about 128 μm; less than about 129 μm; less than about 130 μm; less than about 131 μm; less than about 132 μm; less than about 133 μm; less than about 134 μm; less than about 135 μm; less than about 136 μm; less than about 137 μm; less than about 138 μm; less than about 139 μm; less than about 140 μm; less than about 141 μm; less than about 142 μm; less than about 143 μm; less than about 144 μm; less than about 145 μm; less than about 146 μm; less than about 147 μm; less than about 148 μm; less than about 149 μm; or less than about 150 μm. As another example, the inner foveal thickness can be less than about 73 μm. As another example, the inner foveal thickness can be less than any value between about 1 μm and about 150 μm. Recitation of each of these discrete values is understood to include ranges between each value. Recitation of each range is understood to include discrete values within the range.

In some embodiments, an outer foveal thickness can be measured. For example, the outer fovial thickness can be less than about 1 μm; less than about 2 μm; less than about 3 μm; less than about 4 μm; less than about 5 μm; less than about 6 μm; less than about 7 μm; less than about 8 μm; less than about 9 μm; less than about 10 μm; less than about 11 μm; less than about 12 μm; less than about 13 μm; less than about 14 μm; less than about 15 μm; less than about 16 μm; less than about 17 μm; less than about 18 μm; less than about 19 μm; less than about 20 μm; less than about 21 μm; less than about 22 μm; less than about 23 μm; less than about 24 μm; less than about 25 μm; less than about 26 μm; less than about 27 μm; less than about 28 μm; less than about 29 μm; less than about 30 μm; less than about 31 μm; less than about 32 μm; less than about 33 μm; less than about 34 μm; less than about 35 μm; less than about 36 μm; less than about 37 μm; less than about 38 μm; less than about 39 μm; less than about 40 μm; less than about 41 μm; less than about 42 μm; less than about 43 μm; less than about 44 μm; less than about 45 μm; less than about 46 μm; less than about 47 μm; less than about 48 μm: less than about 49 μm; less than about 50 μm; less than about 51 μm; less than about 52 μm; less than about 53 μm; less than about 54 μm; less than about 55 μm; less than about 56 μm: less than about 57 μm; less than about 58 μm; less than about 59 μm; less than about 60 μm; less than about 61 μm; less than about 62 μm; less than about 63 μm; less than about 64 μm; less than about 65 μm; less than about 66 μm; less than about 67 μm; less than about 68 μm; less than about 69 μm: less than about 70 μm; less than about 71 μm; less than about 72 μm; less than about 73 μm; less than about 74 μm; less than about 75 μm; less than about 76 μm; less than about 77 μm; less than about 78 μm; less than about 79 μm; less than about 80 μm; less than about 81 μm; less than about 82 μm; less than about 83 μm; less than about 84 μm; less than about 85 μm; less than about 86 μm; less than about 87 μm; less than about 88 μm; less than about 89 μm; less than about 90 μm; less than about 91 μm; less than about 92 μm; less than about 93 μm; less than about 94 μm; less than about 95 μm; less than about 96 μm; less than about 97 μm; less than about 98 μm; less than about 99 μm; less than about 100 μm; less than about 101 μm; less than about 102 μm; less than about 103 μm; less than about 104 μm; less than about 105 μm; less than about 106 μm; less than about 107 μm; less than about 108 μm; less than about 109 μm; less than about 110 μm; less than about 111 μm; less than about 112 μm; less than about 113 μm; less than about 114 μm; less than about 115 μm: less than about 116 μm; less than about 117 μm: less than about 118 μm; less than about 119 μm; less than about 120 μm; less than about 121 μm; less than about 122 μm; less than about 123 μm; less than about 124 μm; less than about 125 μm; less than about 126 μm; less than about 127 μm; less than about 128 μm; less than about 129 μm; less than about 130 μm; less than about 131 μm; less than about 132 μm; less than about 133 μm; less than about 134 μm; less than about 135 μm; less than about 136 μm; less than about 137 μm; less than about 138 μm; less than about 139 μm; less than about 140 μm; less than about 141 μm; less than about 142 μm; less than about 143 μm; less than about 144 μm; less than about 145 μm; less than about 146 μm; less than about 147 μm; less than about 148 μm; less than about 149 μm; less than about 150 μm; less than about 151 μm; less than about 152 μm; less than about 153 μm; less than about 154 μm; less than about 155 μm; less than about 156 μm; less than about 157 μm; less than about 158 μm; less than about 159 μm; less than about 160 μm; less than about 161 μm; less than about 162 μm; less than about 163 μm; less than about 164 μm; less than about 165 μm; less than about 166 μm; less than about 167 μm; less than about 168 μm; less than about 169 μm; less than about 170 μm; less than about 171 μm; less than about 172 μm; less than about 173 μm; less than about 174 μm; less than about 175 μm; less than about 176 μm; less than about 177 μm; less than about 178 μm; less than about 179 μm; less than about 180 μm; less than about 181 μm; less than about 182 μm; less than about 183 μm; less than about 184 μm; less than about 185 μm; less than about 186 μm; less than about 187 μm; less than about 188 μm; less than about 189 μm; less than about 190 μm; less than about 191 μm; less than about 192 μm; less than about 193 μm; less than about 194 μm; less than about 195 μm; less than about 196 μm; less than about 197 μm; less than about 198 μm; less than about 199 μm; or less than about 200 μm. As another example, the outer foveal thickness can be less than about 190 μm. As another example, the outer foveal thickness can be less than any value between about 1 μm and about 200 μm. Recitation of each of these discrete values is understood to include ranges between each value. Recitation of each range is understood to include discrete values within the range.

In some embodiments, a total foveal thickness can be measured. For example, the total fovial thickness can be less than about 1 μm; less than about 2 μm; less than about 3 μm; less than about 4 μm; less than about 5 μm; less than about 6 μm; less than about 7 μm; less than about 8 μm; less than about 9 μm; less than about 10 μm; less than about 11 μm; less than about 12 μm; less than about 13 μm; less than about 14 μm; less than about 15 μm; less than about 16 μm; less than about 17 μm; less than about 18 μm; less than about 19 μm; less than about 20 μm; less than about 21 μm; less than about 22 μm; less than about 23 μm; less than about 24 μm; less than about 25 μm; less than about 26 μm; less than about 27 μm; less than about 28 μm; less than about 29 μm; less than about 30 μm; less than about 31 μm; less than about 32 μm; less than about 33 μm; less than about 34 μm; less than about 35 μm; less than about 36 μm; less than about 37 μm; less than about 38 μm; less than about 39 μm: less than about 40 μm; less than about 41 μm; less than about 42 μm; less than about 43 μm; less than about 44 μm; less than about 45 μm; less than about 46 μm; less than about 47 μm; less than about 48 μm; less than about 49 μm; less than about 50 μm; less than about 51 μm; less than about 52 μm; less than about 53 μm; less than about 54 μm; less than about 55 μm; less than about 56 μm; less than about 57 μm; less than about 58 μm; less than about 59 μm; less than about 60 μm; less than about 61 μm; less than about 62 μm; less than about 63 μm; less than about 64 μm; less than about 65 μm; less than about 66 μm; less than about 67 μm; less than about 68 μm; less than about 69 μm; less than about 70 μm; less than about 71 μm; less than about 72 μm; less than about 73 μm; less than about 74 μm; less than about 75 μm; less than about 76 μm; less than about 77 μm; less than about 78 μm; less than about 79 μm; less than about 80 μm; less than about 81 μm; less than about 82 μm; less than about 83 μm; less than about 84 μm; less than about 85 μm; less than about 86 μm; less than about 87 μm; less than about 88 μm: less than about 89 μm; less than about 90 μm; less than about 91 μm; less than about 92 μm; less than about 93 μm; less than about 94 μm; less than about 95 μm; less than about 96 μm; less than about 97 μm; less than about 98 μm; less than about 99 μm; less than about 100 μm; less than about 101 μm; less than about 102 μm; less than about 103 μm; less than about 104 μm; less than about 105 μm; less than about 106 μm; less than about 107 μm; less than about 108 μm; less than about 109 μm; less than about 110 μm; less than about 111 μm; less than about 112 μm; less than about 113 μm; less than about 114 μm; less than about 115 μm; less than about 116 μm; less than about 117 μm; less than about 118 μm; less than about 119 μm; less than about 120 μm; less than about 121 μm; less than about 122 μm; less than about 123 μm; less than about 124 μm; less than about 125 μm; less than about 126 μm; less than about 127 μm; less than about 128 μm; less than about 129 μm; less than about 130 μm; less than about 131 μm: less than about 132 μm; less than about 133 μm: less than about 134 μm; less than about 135 μm; less than about 136 μm; less than about 137 μm; less than about 138 μm; less than about 139 μm; less than about 140 μm; less than about 141 μm; less than about 142 μm; less than about 143 μm; less than about 144 μm; less than about 145 μm; less than about 146 μm; less than about 147 μm: less than about 148 μm; less than about 149 μm: less than about 150 μm; less than about 151 μm; less than about 152 μm; less than about 153 μm; less than about 154 μm; less than about 155 μm; less than about 156 μm; less than about 157 μm; less than about 158 μm; less than about 159 μm; less than about 160 μm; less than about 161 μm; less than about 162 μm; less than about 163 μm; less than about 164 μm; less than about 165 μm; less than about 166 μm; less than about 167 μm; less than about 168 μm; less than about 169 μm; less than about 170 μm; less than about 171 μm; less than about 172 μm; less than about 173 μm; less than about 174 μm; less than about 175 μm; less than about 176 μm; less than about 177 μm; less than about 178 μm; less than about 179 μm; less than about 180 μm; less than about 181 μm; less than about 182 μm: less than about 183 μm; less than about 184 μm; less than about 185 μm; less than about 186 μm; less than about 187 μm; less than about 188 μm; less than about 189 μm; less than about 190 μm; less than about 191 μm; less than about 192 μm; less than about 193 μm; less than about 194 μm; less than about 195 μm; less than about 196 μm; less than about 197 μm; less than about 198 μm; less than about 199 μm; less than about 200 μm; less than about 201 μm; less than about 202 μm; less than about 203 μm; less than about 204 μm; less than about 205 μm; less than about 206 μm; less than about 207 μm; less than about 208 μm; less than about 209 μm; less than about 210 μm; less than about 211 μm; less than about 212 μm; less than about 213 μm; less than about 214 μm; less than about 215 μm; less than about 216 μm; less than about 217 μm; less than about 218 μm; less than about 219 μm; less than about 220 μm; less than about 221 μm; less than about 222 μm; less than about 223 μm; less than about 224 μm; less than about 225 μm; less than about 226 μm; less than about 227 μm; less than about 228 μm; less than about 229 μm; less than about 230 μm; less than about 231 μm; less than about 232 μm; less than about 233 μm; less than about 234 μm; less than about 235 μm; less than about 236 μm; less than about 237 μm; less than about 238 μm; less than about 239 μm; less than about 240 μm; less than about 241 μm; less than about 242 μm; less than about 243 μm; less than about 244 μm; less than about 245 μm; less than about 246 μm; less than about 247 μm; less than about 248 μm; less than about 249 μm; less than about 250 μm; less than about 251 μm; less than about 252 μm; less than about 253 μm; less than about 254 μm; less than about 255 μm; less than about 256 μm; less than about 257 μm; less than about 258 μm; less than about 259 μm; less than about 260 μm; less than about 261 μm; less than about 262 μm; less than about 263 μm; less than about 264 μm; less than about 265 μm; less than about 266 μm; less than about 267 μm; less than about 268 μm; less than about 269 μm; less than about 270 μm; less than about 271 μm; less than about 272 μm; less than about 273 μm; less than about 274 μm; less than about 275 μm: less than about 276 μm; less than about 277 μm: less than about 278 μm; less than about 279 μm; less than about 280 μm; less than about 281 μm; less than about 282 μm; less than about 283 μm; less than about 284 μm; less than about 285 μm; less than about 286 μm; less than about 287 μm; less than about 288 μm; less than about 289 μm; less than about 290 μm; less than about 291 μm; less than about 292 μm; less than about 293 μm; less than about 294 μm; less than about 295 μm; less than about 296 μm; less than about 297 μm; less than about 298 μm; less than about 299 μm; less than about 300 μm; less than about 301 μm; less than about 302 μm; less than about 303 μm; less than about 304 μm; less than about 305 μm; less than about 306 μm; less than about 307 μm; less than about 308 μm; less than about 309 μm; less than about 310 μm; less than about 311 μm; less than about 312 μm; less than about 313 μm; less than about 314 μm; less than about 315 μm; less than about 316 μm; less than about 317 μm; less than about 318 μm; less than about 319 μm; less than about 320 μm; less than about 321 μm; less than about 322 μm; less than about 323 μm; less than about 324 μm; less than about 325 μm; less than about 326 μm; less than about 327 μm; less than about 328 μm; less than about 329 μm; less than about 330 μm; less than about 331 μm; less than about 332 μm; less than about 333 μm; less than about 334 μm; less than about 335 μm; less than about 336 μm; less than about 337 μm; less than about 338 μm; less than about 339 μm; less than about 340 μm; less than about 341 μm; less than about 342 μm; less than about 343 μm; less than about 344 μm; less than about 345 μm; less than about 346 μm; less than about 347 μm; less than about 348 μm; less than about 349 μm; less than about 350 μm; less than about 351 μm; less than about 352 μm; less than about 353 μm; less than about 354 μm; less than about 355 μm; less than about 356 μm; less than about 357 μm; less than about 358 μm; less than about 359 μm; less than about 360 μm; less than about 361 μm; less than about 362 μm; less than about 363 μm; less than about 364 μm; or less than about 365 μm. As another example, the total fovial thickness can be less than about 260 μm. As another example, the total foveal thickness can be less than any value between about 1 μm and about 365 μm. Recitation of each of these discrete values is understood to include ranges between each value. Recitation of each range is understood to include discrete values within the range.

Optical Coherence Tomography (OCT) and Oct Angiography (OCTA)

As described herein, optical coherence tomography angiography (OCTA) uses high speed OCT scanning to analyze signal decorrelation between scans, separating stationary structures from those in motion (e.g., red blood cells). As described herein, the vascular changes can be detected by OCTA. As described herein, the retinal thickness, including various retinal layers, can be detected by conventional OCT or OCTA.

Therapeutic Methods

As described herein, optical coherence tomography (OCT) and optical coherence tomography angiography (OCTA) can offer a noninvasive, cost-efficient and rapid means to screen individuals for pre-clinical neurodegenerative disease (e.g., Alzheimer's disease), and can better identify individuals for whom more expensive and invasive biomarker testing or early therapeutic intervention is justified. As such, a subject with significantly increased foveal avascular zone (FAZ) area or decreased inner foveal thickness (e.g., compared to a control or a standard) can receive further testing, early therapeutic intervention, or preventative therapy. Pharmacotherapeutic agents designed to treat or prevent neuronal loss and brain atrophy can be administered at earlier stages.

Conventional treatments and diagnostics can be used after the disclosed OCT and OCTA tests. Such conventional neurodegenerative disease treatment and diagnoses processes are known in the art (see e.g., F1000Research 2018, 7(F1000 Faculty Rev):1161) (e.g., cholinesterase inhibitors (such as Donepezil, Rivastigmine, Galantamine), gluatamate regulators or NMDA antagonist (such as memantine), neurotrophic compounds). Except as otherwise noted herein, therefore, the process of further testing and therapy can be carried out in accordance with such processes.

Also provided is a process of treating a neurodegenerative disease in a subject in need administration of a therapeutically effective amount of a therapeutic agent, so as to substantially inhibit a neurodegenerative disease, slow the progress of a neurodegenerative disease, or limit the development of a neurodegenerative disease.

Methods described herein are generally performed on a subject in need thereof. A subject in need of the therapeutic methods described herein can be a subject having, diagnosed with, suspected of having, or at risk for developing a neurodegenerative disease. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

Generally, a safe and effective amount of a therapeutic agent is, for example, that amount that would cause the desired therapeutic effect in a subject while minimizing undesired side effects. In various embodiments, an effective amount of a therapeutic agent described herein can a neurodegenerative disease

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of a therapeutic agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to a neurodegenerative disease.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a therapeutic agent can occur as a single event or over a time course of treatment. For example, a therapeutic agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for neurodegenerative disease.

A therapeutic agent can be administered simultaneously or sequentially with another agent, such as an antibiotic, an anti-inflammatory, or another agent. For example, a therapeutic agent can be administered simultaneously with another agent, such as an antibiotic or an anti-inflammatory. Simultaneous administration can occur through administration of separate compositions, each containing one or more of a therapeutic agent, an antibiotic, an anti-inflammatory, or another agent. Simultaneous administration can occur through administration of one composition containing two or more of a therapeutic agent, an antibiotic, an anti-inflammatory, or another agent. A therapeutic agent can be administered sequentially with an antibiotic, an anti-inflammatory, or another agent. For example, a therapeutic agent can be administered before or after administration of an antibiotic, an anti-inflammatory, or another agent.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent decrease toxicity due to high initial doses of the agent; alter the immunogenicity of the agent; decrease dosage frequency, improve taste of the product; or improve shelf life of the product.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Association of Preclinical Alzheimer Disease with Optical Coherence Tomographic Angiography Findings

The following example describes the detection of Alzheimer's disease (AD) in pre-symptomatic subjects. This example suggests that cognitively normal subjects with pre-clinical Alzheimer's disease have retinal abnormalities in addition to architectural alterations, and that these changes occur at earlier stages of Alzheimer's disease than has previously been demonstrated.

Here, study participants with biomarker-positive findings for preclinical Alzheimer disease were found to have retinal microvascular alterations detectable by optical coherence tomographic angiography (OCTA) compared with control individuals with biomarker-negative findings. In this single-center, case-control study, the foveal avascular zone was larger in participants with preclinical Alzheimer disease determined by the presence of β-amyloid biomarkers (mean [SD], 0.364 [0.095] mm²) compared with those without preclinical Alzheimer disease (mean [SD], 0.275 [0.060] mm²). Foveal avascular zone enlargement can offer a noninvasive, cost-efficient, and rapid screen to identify preclinical Alzheimer disease.

Biomarker testing for asymptomatic, preclinical Alzheimer disease (AD) is invasive and expensive. Optical coherence tomographic angiography (OCTA) is a noninvasive technique that allows analysis of retinal and microvascular anatomy, which is altered in early-stage AD.

The objective of this study was to determine whether OCTA can detect early retinal alterations in cognitively normal study participants with preclinical AD diagnosed by criterion standard biomarker testing.

This case-control study included 32 participants recruited from the Charles F. and Joanne Knight Alzheimer Disease Research Center, Washington University in St Louis, St Louis, Mo. Results of extensive neuropsychometric testing determined that all participants were cognitively normal. Participants underwent positron emission tomography and/or cerebral spinal fluid testing to determine biomarker status. Individuals with prior ophthalmic disease, media opacity, diabetes, or uncontrolled hypertension were excluded. Data were collected from Jul. 1, 2016, through Sep. 30, 2017, and analyzed from Jul. 30, 2016, through Dec. 31, 2017.

Main Outcomes and Measures

Automated measurements of retinal nerve fiber layer thickness, ganglion cell layer thickness, inner and outer foveal thickness, vascular density, macular volume, and foveal avascular zone were collected using an OCTA system from both eyes of all participants. Separate model III analyses of covariance were used to analyze individual data outcome.

Results

Fifty-eight eyes from 30 participants (53% female; mean [SD] age, 74.5 [5.6] years; age range, 62-92 years) were included in the analysis. One participant was African American and 29 were white. Fourteen participants had biomarkers positive for AD and thus a diagnosis of preclinical AD (mean [SD] age, 73.5 [4.7] years); 16 without biomarkers served as a control group (mean [SD] age, 75.4 [6.6] years). The foveal avascular zone was increased in the biomarker-positive group compared with controls (mean [SD], 0.364 [0.095] vs 0.275 [0.060] mm²; P=0.002). Mean (SD) inner foveal thickness was decreased in the biomarker-positive group (66.0 [9.9] vs 75.4 [10.6] μm; P=0.03).

Conclusions and Relevance

This study suggests that cognitively healthy individuals with preclinical AD have retinal microvascular abnormalities in addition to architectural alterations and that these changes occur at earlier stages of AD than has previously been demonstrated. Longitudinal studies in larger cohorts are needed to determine whether this finding has value in identifying predinical AD.

Introduction

Alzheimer disease (AD) is the most common form of dementia, affecting an estimated 5.4 million US residents.¹ The pathophysiologic changes of AD involve loss of neurons, brain atrophy, extracellular deposition of β-amyloid (Aβ) plaques, and intracellular accumulation of neurofibrillary tangles.^{2,3} Unfortunately, the classic clinical symptoms of AD, including progressive memory loss and behavioral changes, are only apparent after massive, irreversible neuronal loss has occurred. Preclinical AD is a recently recognized period in which the key pathophysiologic changes are underway within the brain, but symptoms have not yet become apparent.⁴

Preclinical AD can be diagnosed based on the presence of clinically validated biomarkers measuring amyloid burden within the central nervous system. Carbon 11-labeled Pittsburgh Compound B (PiB) (N-methyl-[¹¹C]2-(4′-methylaminophenyl)-6-hydroxybenzothiazole; not commercially available)⁵ and fluorine 18-labeled florbetapir (¹⁸F-AV-45; Amyvid) compounds bind amyloid protein within central nervous system tissue and can estimate disease burden when viewed by positron emission tomography (PET).⁵⁻⁹ In addition, levels of Aβ42 and τ protein in the cerebrospinal fluid (CSF) can be quantified in samples acquired by lumbar puncture.^{2,10,11}

Both tests for biomarker status have especially high negative predictive value in assessing the risk of developing clinically detectable AD.^{10,12,13} In addition, both tests have been validated in long-term longitudinal studies to estimate onset of clinical dementia,¹⁴ such that positive findings for either test is considered diagnostic of preclinical AD.¹¹ Although these methods are useful in assessing individuals at risk for AD, they are expensive, time-consuming, invasive, and difficult to implement in routine clinical screening and care.

Recent data have suggested that AD is also marked by vascular dysfunction, although whether the dysfunction is secondary or contributes to the Aβ accumulation is unclear.¹⁵ In the retina specifically, venous narrowing and reduced blood flow have been established in individuals with AD¹⁶⁻¹⁸ and mild cognitive impairment (MCI).¹⁹ A small study using optical coherence tomographic angiography (OCTA) to compare patients with MCI and those with advanced AD²⁰ suggested decreased density of the deep vascular plexus specifically. However, determination of disease status was based on results of neuropsychiatric testing (e.g., Mini-Mental State Examination) rather than objective biomarker status, which has been shown to be inaccurate in estimating conversion from MCI to dementia,²¹ because it is influenced by other factors such as socioeconomic status, level of education, and presence of confounding neuropsychiatric disorders such as depression and stroke.²²

Because clinical trials are under way to evaluate new drugs designed to prevent neuronal loss, it is imperative to be able to identify which individuals with preclinical AD would benefit from potential therapy. Currently accepted testing methods are expensive and invasive. In this study, we evaluated whether OCTA technology has the potential to characterize early retinal architecture and vascular changes in individuals with preclinical AD.

Methods: Study Participants

Cognitively normal study participants were recruited from the Charles F. and Joanne Knight Alzheimer Disease Research Center (ADRC) of Washington University in St Louis, St Louis, Mo. Study participants were volunteers in the Memory and Aging Project of the ADRC. The study design was approved by the institutional review board of Washington University in St Louis at the Human Research Protection Office and adhered to the tenets of the Declaration of Helsinki.²³ Risks and benefits were discussed with each individual, and written informed consent was obtained before beginning the ophthalmologic examination.

Data were collected from Jul. 1, 2016, through Sep. 30, 2017. Inclusion criteria required a Clinical Dementia Rating classification of 0 (no evidence of dementia). The Clinical Dementia Rating is a 5-point scale used to characterize 6 domains of cognitive function and performance to evaluate Alzheimer type dementia, including memory, orientation, judgment and problem solving, community affairs, home and hobbies, and personal care, based on an extensive battery of neuropsychometric tests (TABLE 1).

TABLE 1
Neuropsychometric Tests That Study Participants
Completed to Create the Clinical Dementia Rating
(CDR). All participants received a score of zero,
or no evidence of cognitive impairment.
Clinical Core Psychometric Battery

UDS 3 Psychometric Tests
Montreal Cognitive Assessment (MoCA)
Craft Story 21 Recall, Immediate and Delayed
Benson Complex Figure: Copy, Recall and Recognition
Number Span Test: Forward & Backwards
Category Fluency (Animals, Vegetables)
Trailmaking A and B
Multilingual Naming Test (MINT)
Verbal Fluency for Letters F & L
Additional HASD Psychometric Tests
Mini Mental State Examination (MMSE)
Wechsler Memory Scale: Assoc Learning & Mental
Control
Wechsler AIS: Block Design & Information
Wechsler AIS - Revised: Digit Symbol Substitution
Wechsler AIS III: Letter Number Sequencing
Free and Cued Selective Reminding Test
Switching Task (consonant vowel odd and even,
CVOE)
Simon Task
Stroop Color Only Task
Stroop Switch
Tapping Task
Handedness (at entry only)
Literacy (Slosson Oral Reading Test, Revised; at entry)

Additional inclusion criteria consisted of completion of PET imaging for PiB or ¹⁸F-AV-45 compound or CSF analysis of Aβ42 protein level within 1 year of recruitment; many participants underwent both tests. Biomarker status was kept by the ADRC during data collection stage so that testing and data gathering were performed in a masked manner. Additional data regarding age, sex, self-reported ethnicity, and medical history were collected from a review of the medical records. Information on family history or genetic testing (such as APOE4 allele status) was not collected in this study.

Exclusion criteria included previously diagnosed, clinically apparent AD. Additional exclusion criteria consisted of a known history of glaucoma or age-related macular degeneration; intraocular pressure of 22 mg Hg or higher dense media opacity precluding measurement; history of ocular trauma or concomitant ocular diseases, including previous retinal disease; presence of significant refractive error (more than 5 diopters [D] of spherical equivalent refraction or 3 D of astigmatism); and previous retinal laser therapy. Additional medical exclusion criteria included diabetes and uncontrolled hypertension.

Methods: Study Procedures

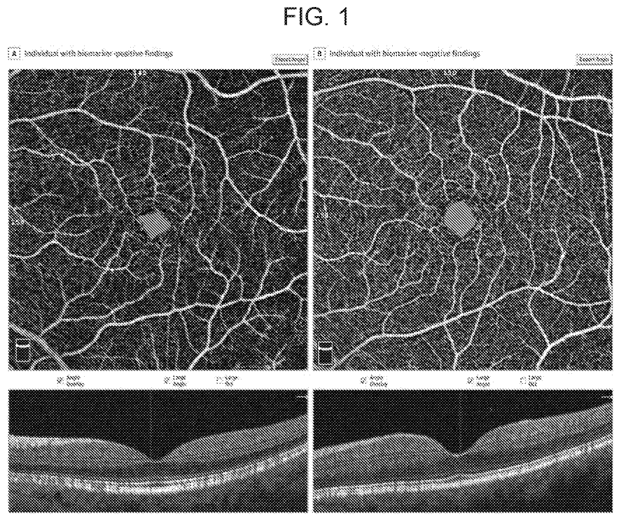

All participants received a complete neuro-ophthalmic examination, including standard assessment of Snellen visual acuity, color perception using Ishihara color plates, ocular motility, intraocular pressure, refractive status, and examination of the anterior segment and dilated fundus. Optical coherence tomographic imaging of the optic disc and macula and OCTA were performed using the Avanti Optovue OCTA system (Optovue, Inc). Measurements were automated using the manufacturer's software (Optovue RTVue) from 6 OCT images per eye and thus collected in an objective manner. Although the software reproducibility has been substantiated in measuring central subfield thickness in diabetic macular edema,²⁴ each data point was reviewed by two of us (B.E.O. and N.K.) to evaluate for potential confounding pathologic findings (e.g., optic nerve head drusen) and subjective appropriateness of the measurements. Data outcomes collected included total and temporal retinal nerve fiber layer thickness; ganglion cell layer thickness; macular volume; inner, outer, and total foveal thickness; total macular, foveal, and parafoveal vascular density; and foveal avascular zone (FAZ) area (see e.g., FIG. 1).

Methods: Data Analysis

Data were analyzed from Jul. 30, 2016, through Dec. 31, 2017. Data outcomes measured on a ratio scale were analyzed using mixed-effects analysis of covariance, whereas data outcomes measured on a percentage scale were analyzed using mixed-effects generalized linear models (GLIMMIX in SAS software; SAS, Inc). Data points for each eye were treated as repeated measurements for the study participant. Separate analyses were run for CSF alone and PET alone and group analysis to compare all participants with at least 1 positive biomarker finding with participants without either biomarker. Age was included as a covariate in the models. Intraocular pressure was also included as a covariate in analyzing retinal nerve fiber layer and ganglion cell layer data. Two-sided P values were generated using SAS software (version 9.4), and these P values were not adjusted because comparisons were not made between the CSF group, PET group, or combined CSF-PET biomarker group.

Results: Descriptive Statistics

A total of 32 study participants were recruited through the Washington University in St Louis ADRC. One patient was excluded owing to suspected undiagnosed normal tension glaucoma based on an increased cup-disc ratio; another was excluded owing to the presence of bilateral optic nerve head drusen. One eye was excluded owing to a full-thickness macular hole and another for vitreomacular traction causing distortion of the retinal architecture. Four images were excluded owing to motion artifact or segmentation error; an additional 6 images were excluded owing to poor automated mapping that did not accurately represent the optic nerve disc or FAZ. Data were collected from 58 eyes of 30 participants (16 women [53%] and 14 men [47%]; mean [SD] age, 74.5 [5.6] years; age range, 62-92 years) for inclusion in the analysis.

Mean (SD) age of participants with biomarker-positive status was 73.5 (4.7) years; of participants with biomarker-negative status, 75.2 (6.6) years. One participant was African American; the remainder reported white race. Among the biomarker-negative group, 10 of 16 (62%) were women; among the biomarker-positive group, 6 of 14 (43%) were women. Common comorbidities included medically controlled hypertension, hyperlipidemia, and depression.

Results: PET Scan Biomarkers

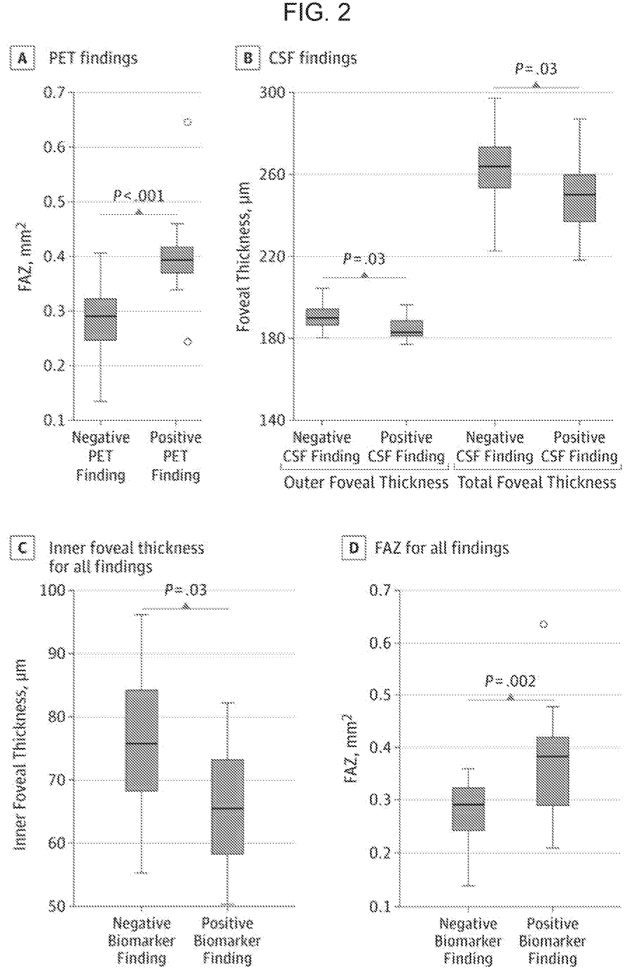

Twenty-seven individuals completed PET scanning for PiB or ¹⁸F-AV-45 binding. Of these, 7 individuals had positive findings for preclinical AD. The mean (SD) age of the PET-negative group was 73.2 (4.6) years; of the PET-positive group, 76.4 (7.6) years old. Mean (SD) FAZ was larger in participants with PET-positive status (0.398 [0.066] mm²) compared with PET-negative controls (0.288 [0.0915] mm²; P<0.001) (see e.g., FIG. 2A).

Results: CSF Biomarker

Twenty-eight individuals completed CSF sampling and analysis. Ten had Aβ42-positive findings and 18 had Aβ42-negative findings. Mean (SD) age of the Aβ42-positive group was 75.7 (7.2) years; of the Aβ42-negative group, 73.1 (4.4) years. Outer foveal measurements were thinner in the Aβ42-positive group (180.8 [8.8] μm) than the Aβ42-negative group (189.3 [10.0] μm; P=0.03) (see e.g., FIG. 2B), as was total foveal thickness (245.9 [16.6] vs 263.0 [17.4] μm; P=0.03) (see e.g., FIG. 2B).

Results: All Biomarker-Positive Findings

Additional analysis was performed comparing individuals with results positive for the CSF or the PET biomarker compared with those with negative results. Inner foveal thickness was smaller in the biomarker-positive group (66.0 [9.9] μm) compared with the biomarker-negative group (75.4 [10.6] μm; P=0.03) (see e.g., FIG. 2C). The FAZ was larger in participants with biomarker-positive findings (0.364 [0.095] mm²) compared with those with biomarker-negative findings (0.275 [0.060] mm²; P=0.002) (see e.g., FIG. 2D).

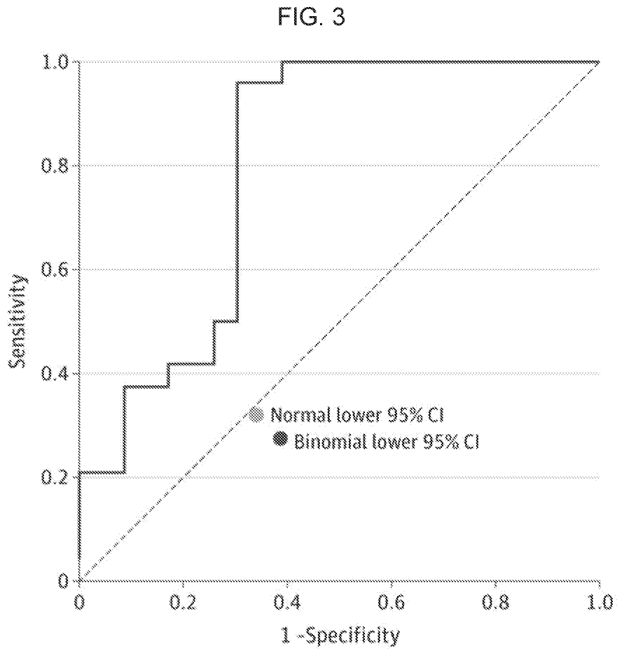

A receiver operating characteristics (ROC) curve was generated for the FAZ in the all-biomarker analysis (see e.g., FIG. 3). The area under the curve was found to be 0.8007 (95% C, 0.6647-0.9367). Given the limited sample size, single, lower 95% CI points were generated for the point along the ROC curve closest to the nondiscriminatory diagonal (50:50) line, assuming normal distribution and binomial distribution (0.26087 and 0.4166).

Discussion

Our data suggest that individuals with biomarker-positive, preclinical AD might have retinal vascular and architectural alterations that are apparent before the onset of clinically detectable cognitive symptoms. This finding may be interpreted to imply that the retina undergoes neuronal loss and vascular modifications far earlier in disease progression than previously thought. A similar phenomenon is seen with AD-associated cerebral neuronal loss, which begins far in advance of symptom onset. However, these findings could be owing to confounding factors unrelated to the FAZ enlargement, and longitudinal studies in larger cohorts are needed to determine whether this finding has value in identifying preclinical AD.

Our findings of inner foveal thinning in participants with biomarker-positive test results are consistent with those of prior studies using traditional OCT technology and early autopsy studies.²⁵⁻²⁷ Although the difference between groups for disease status is statistically significant, the considerable overlap in distribution could make these findings difficult to use clinically.

We also observed dropout of vasculature specifically within the fovea, leading to enlargement of the FAZ in the biomarker-positive group. Since 2007, studies^{8,15,28-30} have reported that vascular dysfunction in individuals with MCI and AD leads to cerebral hypoperfusion during AD development. Older in vivo and autopsy data³¹⁻³⁴ demonstrated that AD is associated with deposition of amyloid and collagen within the cerebral capillaries, resulting in cellular apoptosis and vessel dropout. Because retinal and cerebral vasculature are anatomically and physiologically homologous,^{30-33,35} the retinal vasculature may similarly be affected in AD progression; however, our study is observational and does not investigate causative mechanisms.

Another potential explanation for FAZ enlargement in individuals with preclinical AD may be secondary to retinal degeneration from Aβ accumulation within the retina itself. Several studies have demonstrated accumulation of Aβ plaques in the inner retina of postmortem tissue from individuals with AD³⁶⁻³⁹; although a few sources^{37,39} suggested that the accumulation is limited to the superior retinal tissue, most studies did not comment on location of the deposits. However, other studies in human tissues did not identify retinal Aβ,⁴⁰ and still others suggest that τ accumulation may be more significant.^{41,42} A meta-analysis of the current literature published on retinal amyloid plaques ultimately concluded that “the limited number of eligible studies and their methodological heterogeneity make it impossible to come to a conclusion whether pathological retinal Aβ detection is an effective diagnostic tool for AD.”⁴³

The difference in FAZ distribution between individuals with biomarker-positive and biomarker-negative findings (see e.g., FIG. 2D) provides a potentially clinically useful screening tool, if further studies confirm a false-positive rate of less than 40% as suggested by the ROC curve (see e.g., FIG. 3). Despite a promising area under the curve in the ROC with a lower 95% CI of greater than 0.5, larger, longitudinal studies may not validate our findings. If the final outcome confirms the lower 95% CI at the data points closest to the diagonal line, FAZ would prove to be a poor discriminatory marker in screening for preclinical AD.

Although we excluded participants with diabetes and uncontrolled hypertension from this study, we acknowledge that multiple other potential causes for an enlarged FAZ exist and that further assessment of OCTA in the general population is necessary. Despite this possible limitation, our data suggest that OCTA has the potential for rapid, noninvasive, and cost-effective identification of individuals who are likely to have preclinical AD unless these findings are owing to confounding factors unrelated to the FAZ enlargement. As noted, longitudinal studies in larger cohorts are be needed to determine whether this finding has value in identifying preclinical AD.

Strengths and Limitations

Strengths of our study include the use of biomarkers to identify individuals with preclinical AD. Previously published studies rely on the use of neurocognitive testing, namely, the Mini-Mental State Examination, to identify individuals with early dementia; however, a 2015 Cochrane review²¹ concluded that the Mini-Mental State Examination alone, without supporting testing or repetitive testing, could not accurately estimate conversion from MCI to dementia.

The PET and CSF biomarkers have been clinically validated and correlated with postmortem autopsy study findings^{4,6} and have been validated in longitudinal studies as an early diagnostic marker of individuals who will develop clinically significant Alzheimer-type dementia.¹⁴ In a comparative study, both biomarkers were found to be equally accurate in identifying early-stage AD⁴⁴ with a relatively high concordance of approximately 80%.⁴⁵ In our study, 5 participants underwent PET testing and lumbar puncture with conflicting results; in 4, PET findings were negative but CSF findings were positive; in 1, PET findings were positive but CSF findings were negative. Overall, the discordance rate was 15.6%. Although the participants with biomarker-negative findings who had only 1 biomarker available may have been misclassified, more likely these discrepancies are merely associated with the specificity of the individual test and are in line with a low rate of discordance.⁴⁵⁻⁴⁷ As such, any individual with a positive marker was considered to have biomarker-positive findings in the collective analysis.

A limitation of our study is the small sample size, including a limited number of nonwhite individuals. An additional limitation is exclusion of individuals with known vascular disease from our study; we are therefore unable to determine whether these results are translatable to individuals who may have retinal microvascular changes due to other causes. Also, inclusion only of those with preclinical, biomarker-positive disease limits comparison to those with cognitive changes or advanced AD. Recruitment is under way to evaluate individuals with biomarker-positive MCI and more advanced AD and to follow up with individuals with biomarker-positive findings over time for longitudinal evaluation of changes in retinal vasculature.

### CONCLUSIONS

At present, preclinical AD is diagnosable only by invasive, expensive, and time-consuming PET or CSF testing. Our data suggest that OCTA may enable quick, inexpensive, and noninvasive screening for individuals with preclinical AD based on FAZ enlargement. However, these findings could be owing to confounding factors unrelated to the FAZ enlargement. Longitudinal studies in larger cohorts could be done to further support these findings value in identifying preclinical AD, so that these individuals can receive appropriate care.

### REFERENCES

1. Alzheimer's Association. 2016 Alzheimer's disease facts and figures. Alzheimers Dement. 2016; 12(4):459-509.
2. Ballard C, Gauthier S, Corbett A, Brayne C, Aarsland D, Jones E. Alzheimer's disease. Lancet. 2011; 377(9770):1019-1031.
3. Reitz C, Mayeux R. Alzheimer disease: epidemiology, diagnostic criteria, risk factors and biomarkers. Biochem Pharmacol. 2014; 88(4):640-651.
4. Sperling R A, Aisen P S, Beckett L A, et al. Toward defining the preclinical stages of Alzheimer's disease: recommendations from the National Institute on Aging-Alzheimer's Association workgroups on diagnostic guidelines for Alzheimer's disease. Alzheimers Dement. 2011; 7(3):280-292.
5. Klunk W E, Engler H, Nordberg A, et al. Imaging brain amyloid in Alzheimer's disease with Pittsburgh Compound-B. Ann Neurol. 2004: 55(3):306-319.
6. Bacskai B J, Frosch M P, Freeman S H, et al. Molecular imaging with Pittsburgh Compound B confirmed at autopsy: a case report. Arch Neurol. 2007; 64(3):431-434.
7. Wong D F, Rosenberg P B, Zhou Y, et al. In vivo imaging of amyloid deposition in Alzheimer disease using the radioligand 18F-AV-45 (florbetapir [corrected] F 18). J Nucl Med. 2010; 51(6):913-920.
8. Johnson K A, Sperling R A, Gidicsin C M, et al: AV45-A11 study group. Florbetapir (F18-AV-45) PET to assess amyloid burden in Alzheimer's disease dementia, mild cognitive impairment, and normal aging. Alzheimers Dement. 2013; 9(5)(suppl):S72-S83.
9. Clark C M, Schneider J A, Bedell B J, et al; AV45-A07 Study Group. Use of florbetapir-PET for imaging beta-amyloid pathology [published correction appears in JAMA. 2011; 305(11):1096]. JAMA. 2011; 305(3):275-283.
10. Blennow K, Hampel H, Weiner M, Zetterberg H. Cerebrospinal fluid and plasma biomarkers in Alzheimer disease. Nat Rev Neurol. 2010: 6(3):131-144.
11. Scheltens P, Blennow K, Breteler M M B, et al. Alzheimer's disease. Lancet. 2016; 388(10043):505-517.
12. Morris E, Chalkidou A, Hammers A, Peacock J, Summers J, Keevil S. Diagnostic accuracy of (18)F amyloid PET tracers for the diagnosis of Alzheimer's disease: a systematic review and meta-analysis. Eur J Nucl Med Mol Imaging. 2016; 43(2):374-385.
13. Blennow K, Dubois B, Fagan A M, Lewczuk P, de Leon M J, Hampel H. Clinical utility of cerebrospinal fluid biomarkers in the diagnosis of early Alzheimer's disease. Alzheimers Dement. 2015: 11(1):58-69.
14. Roe C M, Fagan A M, Grant E A, et al. Amyloid imaging and CSF biomarkers in predicting cognitive impairment up to 7.5 years later. Neurology. 2013; 80(19):1784-1791.
15. Di Marco L Y, Venneri A, Farkas E, Evans P C, Marzo A, Frangi A F. Vascular dysfunction in the pathogenesis of Alzheimer's disease: a review of endothelium-mediated mechanisms and ensuing vicious circles. Neurobiol Dis. 2015; 82:593-606.
16. Berisha F, Feke G T, Trempe C L, McMeel J W, Schepens C L. Retinal abnormalities in early Alzheimer's disease. Invest Ophthamol Vis Sci. 2007; 48(5):2285-2289.
17. Cheung C Y-L, Ong Y T, Ikram M K, et al. Microvascular network alterations in the retina of patients with Alzheimer's disease. Alzheimers Dement. 2014; 10(2):135-142.
18. Golzan S M, Goozee K, Georgevsky D, et al. Retinal vascular and structural changes are associated with amyloid burden in the elderly: ophthalmic biomarkers of preclinical Alzheimer's disease. Alzheimers Res Ther. 2017; 9(1):13.
19. Feke G T, Hyman B T, Stem R A, Pasquale L R. Retinal blood flow in mild cognitive impairment and Alzheimer's disease. Alzheimers Dement (Amst). 2015; 1(2):144-151.
20. Jiang H, Wei Y, Shi Y, et al. Altered macular microvasculature in mild cognitive impairment and Alzheimer disease [published online Oct. 16, 2017]. J Neuroophthalmol.
21. Arevalo-Rodriguez I, Smailagic N, Roqué I Figuls M, et al. Mini-Mental State Examination (MMSE) for the detection of Alzheimer's disease and other dementias in people with mild cognitive impairment (MCI). Cochrane Database Syst Rev. 2015; (3):CD010783.
22. Raiha I, Isoaho R, Ojanlatva A, Viramo P, Sulkava R, Kivela S L. Poor performance in the Mini-Mental State Examination due to causes other than dementia. Scand J Prim Health Care. 2001; 19(1):34-38.
23. World Medical Association. World Medical Association Declaration of Helsinki: ethical principles for medical research involving human subjects. JAMA. 2013; 310(20):2191-2194.
24. Bressler S B, Edwards A R, Andreoli C M, et al; for the Diabetic Retinopathy Clinical Research Network/Writing Committee. Reproducibility of Optovue RTVue optical coherence tomography retinal thickness measurements and conversion to equivalent Zeiss Stratus metrics in diabetic macular edema. Transl Vis Sci Technol. 2015; 4(1):5.
25. Hinton D R, Sadun A A, Blanks J C, Miller C A. Optio-nerve degeneration in Alzheimer's disease. N Engl J Med. 1986; 315(8):485-487.
26. Blanks J C, Torigoe Y, Hinton D R, Blanks R H. Retinal pathology in Alzheimer's disease, I: ganglion cell loss in foveal/parafoveal retina. Neurobiol Aging. 1996; 17(3):377-384.
27. Blanks J C, Schmidt S Y, Torigoe Y, Porrello K V, Hinton D R, Blanks R H. Retinal pathology in Alzheimer's disease, II: regional neuron loss and glial changes in GCL. Neurobiol Aging. 1996; 17(3):385-395.
28. Park K W, Yoon H J, Kang D-Y, Kim B C, Kim S, Kim J W. Regional cerebral blood flow differences in patients with mild cognitive impairment between those who did and did not develop Alzheimer's disease. Psychiatry Res. 2012; 203(2-3):201-206.
29. Caroli A, Testa C, Geroldi C, et al. Cerebral perfusion correlates of conversion to Alzheimer's disease in amnestic mild cognitive impairment. J Neurol. 2007; 254(12):1698-1707.
30. Nobili F, Frisoni G B, Portet F, et al. Brain SPECT in subtypes of mild cognitive impairment: findings from the DESCRIPA multicenter study. J Neurol. 2008; 255(9):1344-1353.
31. Kalaria R N, Pax A B. Increased collagen content of cerebral microvessels in Alzheimer's disease. Brain Res. 1995; 705(1-2):349-352.
32. De Jong G I, De Vos R A, Steur E N, Luiten P G. Cerebrovascular hypoperfusion: a risk factor for Alzheimer's disease? animal model and postmortem human studies. Ann N Y Acad Sci. 1997:826:56-74.
33. Christov A, Ottman J, Hamdheydari L, Grammas P. Structural changes in Alzheimer's disease brain microvessels. Curr Alzheimer Res. 2008; 5(4):392-395.
34. Farkas E, De Jong G I, de Vos R A, Jansen Steur E N, Luiten P G. Pathological features of cerebral cortical capillaries are doubled in Alzheimer's disease and Parkinson's disease. Acta Neuropathol. 2000; 100(4):395-402.
35. Patton N, Aslam T, Macgillivray T, Pattie A, Deary I J, Dhillon B. Retinal vascular image analysis as a potential screening tool for cerebrovascular disease: a rationale based on homology between cerebral and retinal microvasculatures. J Anat. 2005; 206(4):319-348.
36. Koronyo-Hamaoui M, Koronyo Y, Ljubimov A V, et al. Identification of amyloid plaques in retinas from Alzheimer's patients and noninvasive in vivo optical imaging of retinal plaques in a mouse model. NeuroImage. 2011; 54S1:S204-S217.
37. Koronyo Y, Biggs D, Barron E, et al. Retinal amyloid pathology and proof-of-concept imaging trial in Alzheimer's disease. JCI Insight. 2017; 2(16):93621.
38. Tsai Y, Lu B, Ljubimov A V, et al. Ocular changes in TgF344-AD rat model of Alzheimer's disease. Invest Ophthalmol Vis Sci. 2014; 55(1):523-534.
39. La Morgia C, Ross-Cisneros F N, Koronyo Y, et al. Melanopsin retinal ganglion cell loss in Alzheimer disease. Ann Neurol. 2016; 79(1):90-109.
40. Ho C-Y, Troncoso J C, Knox D, Stark W, Eberhart C G. Beta-amyloid, phospho-tau and alpha-synuclein deposits similar to those in the brain are not identified in the eyes of Alzheimer's and Parkinson's disease patients. Brain Pathol. 2014; 24(1):25-32.
41. Schön C, Hoffmann N A, Ochs S M, et al. Long-term in vivo imaging of fibrillar tau in the retina of P301S transgenic mice. PLoS One. 2012; 7(12):e53547.
42. Chiasseu M, Alarcon-Martinez L, Belforte N, et al. Tau accumulation in the retina promotes early neuronal dysfunction and precedes brain pathology in a mouse model of Alzheimer's disease. Mol Neurodegener. 2017:12(1):58.
43. Jiang J, Wang H, Li W, Cao X, Li C. Amyloid plaques in retina for diagnosis in Alzheimer's patients: a meta-analysis. Front Aging Neurosci. 2016; 8:267.
44. Palmqvist S, Zetterberg H, Mattsson N, et al; Alzheimer's Disease Neuroimaging Initiative; Swedish BioFINDER Study Group. Detailed comparison of amyloid PET and CSF biomarkers for identifying early Alzheimer disease. Neurology. 2015; 85(14):1240-1249.
45. Vlassenko A G, McCue L, Jasielec M S, et al. Imaging and cerebrospinal fluid biomarkers in early preclinical Alzheimer disease. Ann Neurol. 2016; 80(3):379-387.
46. Forlenza O V, Radanovic M, Talib L L, et al. Cerebrospinal fluid biomarkers in Alzheimer's disease: diagnostic accuracy and prediction of dementia. Alzheimers Dement (Amst). 2015; 1(4):455-463.
47. Bloudek L M, Spackman D E, Blankenburg M, Sullivan S D. Review and meta-analysis of biomarkers and diagnostic imaging in Alzheimer's disease. J Alzheimers Dis. 2011; 26(4):627-645.

1. A method of identifying a subject at risk for developing a neurodegenerative disease, comprising measuring a foveal avascular zone (FAZ) area or an inner foveal thickness, an outer foveal thickness, or a total foveal thickness, wherein
the subject does not exhibit cognitive dysfunction; and
the measuring of the foveal avascular zone (FAZ) area or inner, outer, or total foveal thickness is performed using optical coherence tomography (OCT) or optical coherence tomography angiography (OCTA).

2. The method of claim 1, wherein the neurodegenerative disease causes vascular or retinal abnormalities in an eye of the subject.

3. The method of claim 1, wherein the neurodegenerative disease is an amyloid-β-associated neurodegenerative disease.

4. The method of claim 1, wherein the subject not exhibiting cognitive dysfunction is cognitively normal, has a Clinical Dementia Rating (CDR) of 0 or has no evidence of cognitive impairment.

5. The method of claim 1, wherein if FAZ area is increased compared to a control or standard or if the inner foveal thickness, the outer foveal thickness, or the total foveal thickness is decreased compared to a control or standard, the subject is identified as being at risk for developing or having a neurodegenerative disease, wherein the control or standard is obtained from a subject not having a neurodegenerative disease or a preclinical neurodegenerative disease.

6. The method of claim 1, wherein if the measured FAZ area is greater than about 0.3 mm², the subject is identified as being at risk for developing or at risk for having a neurodegenerative disease.

7. The method of claim 1, wherein if the inner foveal thickness is less than about 73 μm, the subject is identified as being at risk for developing or at risk for having a neurodegenerative disease.

8. The method of claim 1, wherein if the outer foveal thickness is less than 190 μm, the subject is identified as being at risk for developing or at risk for having a neurodegenerative disease.

9. The method of claim 1, wherein if the total foveal thickness is less than about 260 μm, the subject is identified as being at risk for developing or at risk for having a neurodegenerative disease.

10. (canceled)

11. The method of claim 1, wherein the neurodegenerative disease is an amyloid-β-associated neurodegenerative disease, preclinical Alzheimer's disease, or dementia.

12. The method of claim 11, wherein the neurodegenerative disease is preclinical Alzheimer's disease.

13. The method of claim 1, wherein the subject is administered early therapeutic intervention to treat or prevent neuronal loss or brain atrophy.

14. The method of claim 1, comprising obtaining a CSF sample from the subject, wherein the CSF sample comprises increased levels of Aβ-42 and tau protein compared to a control or a standard, wherein the control or standard is obtained from a subject not having a preclinical neurodegenerative disease.

15. The method of claim 1, comprising administering a PET imaging agent to a subject selected from Pittsburgh compound and Florbetapir ¹⁸F-AV-45 compound and detecting the PET imaging agent using PET.

16. A method of detecting a preclinical neurodegenerative disease in a subject comprising measuring a foveal avascular zone (FAZ) area or measuring an inner foveal thickness, an outer foveal thickness, or a total foveal thickness, wherein
the subject does not exhibit cognitive dysfunction; and
the measuring of the foveal avascular zone (FAZ) area or inner, outer, or total foveal thickness is performed using optical coherence tomography (OCT) or optical coherence tomography angiography (OCTA).

17. The method of claim 16, wherein the preclinical neurodegenerative disease causes vascular or retinal abnormalities in an eye of the subject.

18. The method of claim 16, wherein the preclinical neurodegenerative disease is an amyloid-β-associated neurodegenerative disease.

19. The method of claim 16, wherein the subject not exhibiting cognitive dysfunction is cognitively normal, has a Clinical Dementia Rating (CDR) of 0 or has no evidence of cognitive impairment.

20. The method of claim 16, wherein an increased FAZ area compared to a control or standard or a decrease of the inner foveal thickness, the outer foveal thickness, or the total foveal thickness, compared to a control or standard, indicates detection of a preclinical neurodegenerative disease, wherein the control or standard is obtained from a subject not having a neurodegenerative disease or a preclinical neurodegenerative disease.

21. The method of claim 16, wherein an FAZ area greater than about 0.3 mm² indicates detection of a preclinical neurodegenerative disease.

22. The method of claim 16, wherein an inner foveal thickness less than about 73 μm indicates detection of a preclinical neurodegenerative disease.

23. The method of claim 16, wherein an outer foveal thickness less than 190 μm indicates detection of a preclinical neurodegenerative disease.

24. The method of claim 16, wherein a total foveal thickness less than about 260 μm indicates detection of a preclinical neurodegenerative disease.

25. (canceled)

26. The method of claim 16, wherein the preclinical neurodegenerative disease is a preclinical amyloid-β-associated neurodegenerative disease, preclinical Alzheimer's disease, or preclinical dementia.

27. The method of claim 26, wherein the preclinical neurodegenerative disease is preclinical Alzheimer's disease.

28. The method of claim 16, wherein the subject is administered early therapeutic intervention to treat or prevent neuronal loss or brain atrophy.

29. The method of claim 16, comprising obtaining a CSF sample from the subject, wherein the CSF sample comprises increased levels of Aβ-42 and tau protein compared to a control or standard, wherein the control or standard is obtained from a subject not having a preclinical neurodegenerative disease.

30. The method of claim 16, comprising administering a PET imaging agent to a subject selected from Pittsburgh compound and Florbetapir ¹⁸F-AV-45 compound and detecting the PET imaging agent using PET.

31. The method of claim 5, wherein the control or standard is selected from measurements from (i) a subject having been administered a PET imaging agent selected from Pittsburgh compound and Florbetapir ¹⁸F-AV-45 compound and detecting the PET imaging agent using PET or (ii) a subject having CSF analysis of Aβ42 protein level and the subject was PET-negative or Aβ42-negative.

32. The method of claim 20, wherein the control or standard is selected from measurements from (i) a subject having been administered a PET imaging agent selected from Pittsburgh compound and Florbetapir ¹⁸F-AV-45 compound and detecting the PET imaging agent using PET or (ii) a subject having CSF analysis of Aβ42 protein level and the subject was PET-negative or Aβ42-negative.
